(19) United States
(12) Patent Application Publication
(10) Pub. No.: US 2014/0277023 A1
(43) Pub. Date: Sep. 18, 2014

(54) FLUID EJECTION DEVICE

(71) Applicant: SEIKO EPSON CORPORATION, Tokyo (JP)

(72) Inventors: Hirokazu SEKINO, Chino-shi (JP); Atsuya HIRABAYASHI, Chino-shi (JP)

(73) Assignee: SEIKO EPSON CORPORATION, Tokyo (JP)

(21) Appl. No.: 14/213,271

(22) Filed: Mar. 14, 2014

(30) Foreign Application Priority Data
Mar. 15, 2013 (JP) 2013-053136

(51) Int. Cl. A61B 17/3203; B05B 1/00

(52) U.S. Cl. CPC A61B 17/3203; B05B 1/00
USPC 606/167; 239/128

(57) ABSTRACT

A fluid ejection device for ejecting fluid includes an ejection tube including an opening to eject the fluid, a pulsation applying part which communicates with the ejection tube and applies pulsation to the fluid, a supply flow path to supply the fluid to the pulsation applying part, and a cooling part to cool the fluid in the supply flow path.

This application claims the benefit of Japanese Patent Application No. 2013-53136, filed on Mar. 15, 2013. The content of the aforementioned application is incorporated herein by reference in its entirety.

### BACKGROUND

1. Technical Field

The present invention relates to a fluid ejection device.

2. Related Art

As a medical apparatus which applies an ejected fluid to an affected part and treats, for example, one disclosed in JP-A-2003-111766 (Patent Literature 1) is known. Patent Literature 1 discloses a technique in which a laser is irradiated to fluid in a catheter to vaporize a part of the fluid and to raise the pressure of the fluid in the catheter, and the fluid is ejected from an opening at the tip of the catheter.

Besides, JP-A-2005-152094, JP-A-2003-500098 and JP-A-7-155335 are examples of related art.

However, in the technique disclosed in Patent Literature 1, since the laser is irradiated to the fluid, the temperature of the fluid rises, and there is a problem that the fluid of suitable temperature may not be ejected to the affected part.

Incidentally, this problem is not limited to the fluid ejection device in which the laser is irradiated to fluid and pulsation is applied to the fluid, and is a problem common to a general fluid ejection device in which when fluid passes through a pulsation applying part for applying pulsation, the temperature of the fluid rises.

Further, in the related art fluid ejection device, miniaturization thereof, cost reduction, resource saving, manufacture facilitation, usability improvement and the like are desired.

### SUMMARY

An advantage of some aspects of the invention is to solve at least a part of the above problems described above and the invention can be implemented as the following aspects.

(1) An aspect of the invention provides a fluid ejection device for ejecting fluid. The fluid ejection device includes an ejection tube including an opening to eject the fluid, a pulsation applying part which communicates with the ejection tube and applies pulsation to the fluid, a supply flow path to supply the fluid to the pulsation applying part, and a cooling part to cool the fluid in the supply flow path. According to this fluid ejection device, the temperature of the fluid supplied to the pulsation applying part can be lowered. According to fluid ejection device of this aspect, the temperature rise of the fluid by the pulsation applying part is suppressed, and the temperature of the ejected fluid can be made to fall within a suitable range.

(2) In the fluid ejection device of the aspect described above, the cooling part may be attachable to and detachable from the supply flow path. According to the fluid ejection device of this aspect, even when the supply flow path is discarded, the cooling part is detached and can be reused.

(3) The fluid ejection device may further include a housing to house the pulsation applying part, and the cooling part may be provided outside the housing. According to the fluid ejection device of this aspect, since the cooling part is provided outside the housing, the cooling part can be easily detached and attached.

(4) In the fluid ejection device of the aspect described above, the cooling part may include a cooling circuit including a Peltier element. According to the fluid ejection device of this aspect, the cooling part can be miniaturized.

(5) In the fluid ejection device of the aspect described above, the pulsation applying part may include a bubble generating part to generate a bubble in the fluid according to an intermittent drive signal. The temperature of the fluid rises by irradiation energy from the bubble generating part. According to the fluid ejection device of this aspect, since the temperature of the fluid supplied to the bubble generating part can be lowered, the temperature rise of the fluid by the bubble generating part is suppressed, and the temperature of the ejected fluid can be made to fall within a suitable range.

(6) Another aspect of the invention provides a medical apparatus including the fluid ejection device of the aspect described above. According to this aspect of the invention, the highly reliable medical apparatus can be provided.

Not all of the plural components of the foregoing aspects of the invention are inevitable, and in order to solve part of or all of the foregoing problems or in order to achieve part of or all of the effects disclosed in the present specification, part of the plural components can be appropriately changed, deleted or substituted by another new component, or part of the limited contents can be deleted. Besides, in order to solve part of or all of the foregoing problems or in order to achieve part of or all of the effects disclosed in the specification, part of or all of the technical features included in one aspect of the invention can be combined with part of or all of the technical features included in another aspect of the invention to obtain one independent form of the invention.

For example, one aspect of the invention can be realized as an apparatus including at least one of the four components, that is, the ejection tube, the pulsation applying part, the supply flow path and the cooling part. That is, this apparatus may include the ejection tube or may not include the ejection tube. Besides, this apparatus may include the pulsation applying part or may not include pulsation applying part. Besides, this apparatus may include the supply flow path or may not include the supply flow path. Besides, this apparatus may include the cooling part or may not include the cooling part.

The ejection tube may be constructed as, for example, an ejection tube including an opening to eject the fluid. The pulsation applying part may be constructed as, for example, a pulsation applying part which communicates with the ejection tube and applies pulsation to the fluid. The supply flow path may be constructed as, for example, a supply flow path to supply the fluid to the pulsation applying part. The cooling part may be constructed as, for example, a cooling part to cool the fluid in the supply flow path. Although such an apparatus can be realized as the fluid ejection device, it can be realized as another apparatus other than the fluid ejection device. According to the forms as described above, at least one of various problems such as miniaturization of the apparatus, cost reduction, resource saving, manufacture facilitation and usability improvement can be solved. Part of or all of the technical features of the respective forms of the foregoing fluid ejection device can be applied to this apparatus.

The invention can be realized in various forms other than the apparatus. For example, the invention can be realized in the form of a method for ejecting a fluid or a manufacturing method of a fluid ejection device.

### BRIEF DESCRIPTION OF THE DRAWINGS

The invention will be described with reference to the accompanying drawings, wherein like numbers reference like elements.

FIG. 1 is an explanation view showing a structure of a fluid ejection device of an embodiment of the invention.

FIG. 2 is an explanatory view showing a structure of a fluid ejection device of a second embodiment.

### DESCRIPTION OF EXEMPLARY EMBODIMENTS

Next, embodiments of the invention will be described in the following sequence.
A. First Embodiment:
B. Second Embodiment:
C. Modified Example:

### A. First Embodiment

FIG. 1 is an explanation view showing a structure of a fluid ejection device 100 of an embodiment of the invention.

The fluid ejection device 100 of this embodiment is a medical apparatus used in a medical institution, and has a function as a scalpel to incise or excise an affected part by ejecting a fluid to the affected part.

The fluid ejection device 100 includes a fluid container 10, a fluid supply mechanism 20, a hand piece 30, a laser oscillation part 40 and a cooling circuit 50. Incidentally, in FIG. 1, the inner structure of a part of the hand piece 30 is enlarged and shown.

The fluid container 10 and the fluid supply mechanism 20 are connected by a connection tube 21, and the fluid supply mechanism 20 and the hand piece 30 are connected by a connection tube 22. In this embodiment, the connection tubes 21 and 22 are made of resin. The hand piece 30 and the laser oscillation part 40 are connected by an optical fiber cable 41.

The fluid container 10 contains physiological saline solution as fluid supplied to the hand piece 30. However, the fluid container 10 may contain, instead of physiological saline solution, another fluid, such as pure water or liquid medicine, which is harmless even when it is ejected to an affected part.

The fluid supply mechanism 20 supplies the fluid contained in the fluid container 10 to the hand piece 30 through the connection tubes 21 and 22. In this embodiment, a pump is used as the fluid supply mechanism 20.

The hand piece 30 is a tool grasped by an operator and is operated, and includes a fluid ejection tube 31, a pulsation applying part 32 and a housing 33. The hand piece 30 ejects the fluid (pulsating flow), to which pulsation is applied, at high speed from an opening 31a (nozzle 31a) of a tip of the fluid ejection tube 31. The operator applies the fluid ejected from the hand piece 30 to a living tissue as an affected part of a patient, and performs treatment such as, for example, incision or excision of the affected part.

The pulsation applying part 32 is provided inside the housing 33, applies pulsation to the fluid, and ejects the pulsating flow from the opening 31a of the fluid ejection tube 31. An inlet flow path 32a, an intermediate flow path 32b and an outlet flow path 32c are formed inside the pulsation applying part 32. The intermediate flow path 32b is a flow path positioned between the inlet flow path 32a and the outlet flow path 32c.

The connection tube 22 is connected to the inlet flow path 32a of the pulsation applying part 32, and the fluid ejection tube 31 is connected to the outlet flow path 32c. Further, the optical fiber cable 41 is connected to the pulsation applying part 32, and an irradiation part 41a of a tip of the optical fiber cable 41 is exposed to the intermediate flow path 32b.

The laser oscillation part 40 irradiates a laser to the fluid in the intermediate flow path 32b through the optical fiber cable 41. In this embodiment, the laser oscillation part irradiates a holmium YAG laser to the fluid in the intermediate flow path 32b. The laser oscillation part 40 may irradiate another kind of laser such as a semiconductor laser, a gas laser or a dye laser. When a continuous oscillation type laser is used, a structure may be made such that for example, a mirror or a shutter is provided in the middle of the laser path, and the laser is irradiated only in a required period.

Since the fluid irradiated by the laser vaporizes and becomes bubbles, the pressure in the flow path of the pulsation applying part 32 abruptly increases. The fluid in the flow path of the pulsation applying part 32 and in the fluid ejection tube 31 is abruptly pushed out, and is ejected from the opening 31a of the fluid ejection tube 31. In this embodiment, the laser oscillation part 40 intermittently irradiates the laser, so that the pulse flow (pulsating flow) is ejected from the opening 31a of the fluid ejection tube 31.

In the fluid ejection device 100 having the above structure, according to this embodiment, the connection tube 22 is provided with the cooling circuit 50 including a Peltier element. The cooling circuit 50 cools the fluid in the connection tube 22, and the cooled fluid is supplied to the pulsation applying part 32. That is, according to this embodiment, since the temperature of the fluid supplied to the pulsation applying part 32 can be previously lowered, the temperature rise of the fluid by the irradiation of the laser in the pulsation applying part 32 is suppressed, and the temperature of the ejected fluid can be made to fall within a suitable range.

Further, according to this embodiment, since the cooled fluid is supplied to the pulsation applying part 32, the pulsation applying part 32 and the irradiation part 41a of the tip of the optical fiber cable 41 can be cooled.

Further, according to this embodiment, the operator confirms the temperature of the fluid ejection tube 31 or the housing 33 and can confirm whether, after pulsation is applied, the fluid falls within the suitable temperature range. For example, when the cooling circuit 50 breaks down and does not operate, since the fluid supplied to the pulsation applying part 32 is not cooled, the temperature of the pulsation applying part 32, the fluid ejection tube 31 and the housing 33 rises. When the operator grasps the hand piece 30 and performs treatment, the operator can notice that the temperature of the fluid ejection tube 31 or the housing 33 rises. That is, the operator notices that the fluid does not fall within the suitable temperature range after pulsation is applied and that the cooling circuit 50 breaks down and does not operate, and the operator can interrupt the treatment.

Incidentally, a structure may be adopted in which a temperature sensor is provided in the fluid ejection tube 31 or the housing 33, whereby it is monitored whether, after pulsation is applied, the fluid falls within the suitable temperature range. Besides, a structure may be adopted in which, after pulsation is applied, when the temperature of the fluid does not fall within the suitable temperature range, an alarm sound is issued or an alarm is displayed on a monitor. Besides, a structure may be adopted in which, after pulsation is applied, when the temperature of the fluid does not fall within the suitable temperature range, the fluid supply mechanism 20 and the laser oscillation part 40 are stopped, and ejecting of the fluid is stopped.

Further, in this embodiment, the cooling circuit 50 can be detached from and attached to the connection tube 22. Accordingly, even when the connection tube 22 is discarded, the cooling circuit 50 is detached and can be reused.

Further, in this embodiment, since the cooling circuit 50 is provided outside the housing 33 of the hand piece 30, the cooling circuit 50 can be easily detached and attached.

As described above, according to this embodiment, since the temperature of the fluid supplied to the pulsation applying part 32 can be lowered by the cooling circuit 50, the temperature rise of the fluid by the pulsation applying part 32 is suppressed, and the temperature of the ejected fluid can be made to fall within the suitable range.

### B. Second Embodiment

FIG. 2 is an explanatory view showing a structure of a fluid ejection device 100b of a second embodiment. A main difference from the first embodiment shown in FIG. 1 is that a temperature sensor 52 is provided in a fluid ejection tube 31, and a control part 54 is provided. The other structure is the same as that of the first embodiment.

The temperature sensor 52 measures the temperature of fluid in the fluid ejection tube 31. The control part 54 performs feedback control of a cooling circuit 50 so that the temperature of the fluid measured by the temperature sensor 52 falls within a previously set temperature range.

According to the second embodiment, the same effects as those of the first embodiment can be obtained, and the temperature of the fluid ejected from the fluid ejection tube 31 can be made to fall within the suitable range.

Incidentally, the temperature sensor 52 may be provided at a place where the temperature of the fluid can be measured after pulsation is applied, or maybe provided between the cooling circuit 50 and the pulsation applying part 32. Besides, a structure may be adopted in which when, after pulsation is applied, the temperature of the fluid does not fall within the suitable temperature range during a specified time period, the fluid supply mechanism 20 and the laser oscillation part 40 are stopped, and ejecting of the fluid is stopped.

### C. Modified Example

Incidentally, the invention is not limited to the above embodiments, and can be carried out in various modes within the scope not departing from the gist thereof. For example, the following modifications can be made.

### Modified Example 1

In the above embodiment, the irradiation part 41a of the tip of the optical fiber cable 41 is provided inside the pulsation applying part 32. The pulsation applying part 32 maybe constructed of a fluid chamber connected to the fluid ejection tube 31 and a piezoelectric element to change the volume of the fluid chamber. When a drive voltage is applied to the piezoelectric element, the temperature of the piezoelectric element rises, and the heat of the piezoelectric element is conducted to the fluid chamber. Thus, even when the pulsation applying part 32 is constructed of the fluid chamber and the piezoelectric element, the temperature of the fluid rises. Accordingly, the invention can be applied also to the fluid ejection device 100 in which the pulsation applying part 32 is constructed of the fluid chamber and the piezoelectric element.

### Modified Example 2

In the above embodiment, the cooling circuit 50 is provided in the connection tube 22 to connect the fluid supply mechanism 20 and the hand piece 30. However, the cooling circuit 50 may be provided in the connection tube 21 to connect the fluid container 10 and the fluid supply mechanism 20. Besides, the cooling circuit 50 may be provided inside the housing 33. When the cooling circuit 50 is provided inside the housing 33, the temperature in the housing 33 can be indirectly lowered through the connection tube 21. Further, the laser oscillation part 40 is provided inside the housing 33, and the cooling circuit 50 may be provided at a position where the cooling circuit contacts the laser oscillation part 40. The cooling circuit 50 contacts the laser oscillation part 40, so that heat generation of the laser oscillation part 40 can be suppressed. Besides, the cooling circuit 50 may be provided in the vicinity of the inlet flow path 32a of the pulsation applying part 32. Besides, the cooling circuit 50 may be constructed not to be detached and attached.

Besides, another cooling mechanism may be used instead of the cooling circuit 50. For example, a container containing ice water may be made to contact the connection tubes 21 and 22. However, according to the cooling circuit 50 including the Peltier element, the apparatus can be miniaturized. Besides, plural cooling circuits 50 may be provided.

### Modified Example 3

In the above embodiment, the fluid ejection device 100 is used as a medical apparatus. However, the fluid ejection device 100 may be used as an apparatus other than the medical apparatus. For example, the fluid ejection device 100 may be used as a cleaning apparatus for removing contamination of a target by applying ejected fluid to the target, or as a drawing apparatus for drawing a character or a picture by ejected fluid.

Also in such an apparatus, when fluid of suitable temperature is required to be ejected to a target, the invention can be applied.

### Modified Example 4

In the above embodiment, liquid is used as the fluid ejected from the fluid ejection device 100. However, gas may be used as the fluid ejected from the fluid ejection device 100.

The invention is not limited to the foregoing embodiments and the modified examples, and can be realized in various structures within the scope not departing from the gist thereof. For example, the technical features in the embodiments and the modified examples corresponding to the technical features in the respective forms recited in the column of SUMMARY can be appropriately replaced or combined in order to solve part of or all of the foregoing problems or in order to achieve part of or all of the foregoing effects. Besides, unless the technical feature is described as being inevitable in the specification, it can be appropriately deleted.

What is claimed is:

1. A fluid ejection device for ejecting fluid, comprising:
an ejection tube including an opening to eject the fluid;
a pulsation applying part which communicates with the ejection tube and applies pulsation to the fluid;
a supply flow path to supply the fluid to the pulsation applying part; and
a cooling part to cool the fluid in the supply flow path.

2. The fluid ejection device according to claim 1, wherein the cooling part is attachable to and detachable from the supply flow path.

3. The fluid ejection device according to claim 2, further comprising a housing to house the pulsation applying part, wherein
the cooling part is provided outside the housing.

4. The fluid ejection device according to claim 1, wherein the cooling part includes a cooling circuit including a Peltier element.

5. The fluid ejection device according to claim 1, wherein the pulsation applying part includes a bubble generating part to generate a bubble in the fluid according to an intermittent drive signal.

6. A medical apparatus comprising a fluid ejection device according to claim 1.

7. A medical apparatus comprising a fluid ejection device according to claim 2.

8. A medical apparatus comprising a fluid ejection device according to claim 3.

9. A medical apparatus comprising a fluid ejection device according to claim 4.

10. A medical apparatus comprising a fluid ejection device according to claim 5.
